(19) United States
(12) Patent Application Publication
(10) Pub. No.: US 2023/0181537 A1
(43) Pub. Date: Jun. 15, 2023

(54) Translation Blockers Repurposed for COVID-19 Therapy

(71) Applicant: The General Hospital Corporation, Boston, MA (US)

(72) Inventors: Jack Rogers, Arlington, MA (US); Catherine Cahill, Arlington, MA (US)

(21) Appl. No.: 17/923,838

(22) PCT Filed: May 7, 2021
(86) PCT No.: PCT/US2021/031405

§ 371 Date: Nov. 7, 2022

Related U.S. Application Data
(60) Provisional application No. 63/021,194, filed on May 7, 2020.

(51) Int. Cl. A61K 31/4184; A61K 31/4745; A61K 31/407; A61K 31/42; A61P 31/14; A61K 31/53

(52) U.S. Cl. CPC A61K 31/4184; A61K 31/42; A61K 31/53; A61K 31/407; A61K 31/4745; A61P 31/14

(57) ABSTRACT

Described herein are methods of treating a subject who has an infection with a coronavirus, e.g., SARS-COV-2, and for reducing risk of infection or severity of infection, with a coronavirus, e.g., SARS-COV-2, the method comprising administering an effective amount of a benzimidazole.

### CLAIM OF PRIORITY

This application claims the benefit of U.S. Provisional Application Serial No. 63/021194, filed on May 7, 2020. The entire contents of the foregoing are incorporated herein by reference.

### TECHNICAL FIELD

Described herein are methods of treating a subject who has an infection with a coronavirus, e.g., SARS-COV-2, and for reducing risk of infection or severity of infection, with a coronavirus, e.g., SARS-COV-2, the method comprising administering an effective amount of a benzimidazole.

### BACKGROUND

Widespread infection with severe acute respiratory syndrome coronavirus 2 (SARS-CoV-2) has caused a global Corona Virus Disease-2019 (COVID-19) pandemic, challenging healthcare and economic systems worldwide. No successful treatments, other than those addressing individual symptoms such as oxygenation, have been identified (Ganesh et al., Clin Epidemiol Glob Health. 2021 April-June; 10: 100694; Graham et al., Ann Clin Transl Neurol. 2021 Mar 23; Lopez-Leon et al., medRxiv. 2021 Jan 30;2021.01.27.21250617; Dharma et al., Clin Microbiol Rev. 2020 Oct; 33(4): e00028-20; Esakanderie et al., Biol Proced Online. 2020; 22: 19; Lotfi et al., Clin Chim Acta. 2020 Sep; 508: 254-266).

### SUMMARY

The present invention is based, at least in part, on the discovery that two CRRM RNA stem loops in the replicase open reading frame of SARS-CoV-2 have high homology with the 5′UTR of amyloid precursor protein (APP), providing a new antiviral target. A number of compounds, including several FDA pre-approved and novel drugs, were previously shown to dose responsively inhibit APP translation via its 5′untranslated region. [6, 7, 46]. The anti COVID-19 activity of drugs that inhibit APP translation, including the APP 5′UTR blocker JTR-009 and analogs thereof, was evaluated, and the results showed that these drugs can reduce infectivity and can thus be used to treat infections, as well as reduce the risk of infection, with SARS-CoV-2.

Thus, provided herein are methods of treating a subject who has an infection with a coronavirus, and for reducing the risk of infection and/or severity of infection (e.g., reducing risk of severe infection). The methods include administering an effective amount of a benzimidazole, e.g., a composition comprising a benzimidazole, e.g., a composition comprising or consisting of a benzimidazole as an active agent. Also provided herein are benzimidazoles, e.g., compositions comprising a benzimidazole, e.g., compositions comprising or consisting of a benzimidazole as an active agent, for use in methods of treating a subject who has an infection with a coronavirus, and for reducing the risk of infection and/or severity of infection. The methods can include administering one or more of the benzimidazoles and/or other agents described herein. In some embodiments, a combination of agents is used, e.g., a combination of a benzimidazole with desferrioxamine, deferriprone, or a vaccine.

In some embodiments, the methods include oral or inhlataional administration of the benzimidazole; in some embodiments, e.g., for reducing risk of infection, administration is by inhalation.

In some embodiments, the coronavirus is SARS-CoV-2.

In some embodiments, the coronavirus comprises a sequence comprising CAGUGCAAGG (SEQ ID NO: 1) or CAGUGU (SEQ ID NO:2). Other viruses comprising these sequences can also be treated using the methods and agents described herein.

In some embodiments, the benzimidazole binds to a sequence comprising CAGUGCAAGG (SEQ ID NO: 1) or CAGUGU (SEQ ID NO:2).

In some embodiments, the benzimidazole is an antihelmintic, e.g., albendazole, fenbendazole, or oxibendazole, or an antacid, e.g., lansoprazole, omeprazole, esomaprazole, or rabeprazole.

In some embodiments, the benzimidazole is JTR-009 or an analog thereof.

In some embodiments, the benzimidazole is BL-1.

Also provided herein are methods for treating a subject who has an infection with a coronavirus, and for reducing the risk of infection and/or severity of infection. The methods include administering an effective amount of an agent selected from Syn-516; A3; Azithromycin; pilocarpine; dimercaptopropanol; tetracycline; paroxetine; tamsulosin; desferrioxamine (DFO); deferriprone; phenserine or posiphen; vindoline and analogs thereof, optionally B1, vincadifformine, vindolinine, (1)-methyl-14,15-didehydroaspidofractinine, Kopsijasminine, or ethyl (1R,9R,16R,17R,18S,21R)-17-hydroxy-2,12-diazahexacyclo[14.2.2.19,12.01,9.03,8.016,21]henicosa-3,5,7,14-tetraene-18-carboxylate; prochlorperazine; diphenylhydramine; netilmycin; mitomycin C; and diptheria toxin. Also provided herein are these agents, e.g., compositions comprising these agents, e.g., compositions comprising or consisting of one or more of these agents as an active agent, for use in methods of treating a subject who has an infection with a coronavirus, and for reducing the risk of infection and/or severity of infection. In some embodiments, a combination of agents is used, e.g., a combination of an agent described herein with desferrioxamine, deferriprone, or a vaccine.

In some embodiments, the coronavirus is SARS-CoV-2.

In some embodiments, the coronavirus comprises a sequence comprising CAGUGCAAGG (SEQ ID NO: 1) or CAGUGU (SEQ ID NO:2). Other viruses comprising these sequences can also be treated using the methods and agents described herein. In some embodiments, the agent binds to a sequence comprising CAGUGCAAGG (SEQ ID NO: 1) or CAGUGU (SEQ ID NO:2).

In some embodiments, the agent is formulated for oral or inhalational administration.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of a predicted pseudo-knot RNA stem loop in the replicase transcript flanked by two CRRM (COVID replicase repeat motifs) RNA targets at +5365 and +17920 in ORF1a/b. Six small boxes show related CAGUGU RNA targets.

FIG. 2 is a schematic illustration of CRRM-1 and -2 and structurally related 5′UTR regions in the mRNAs for the Alzheimer’s-related amyloid precursor protein (APP); Parkinson’s related synuclein alpha (SNCA); and prion protein (PrP). The RNA target is represented twice in the approximately 30 Kb-long COVID-19 RNA genome. There is a marked structural homology between the neurodegenerative 5′UTRs, particularly of APP mRNA specific 5′UTR IRE-Type II and the newly discovered replicase repeat motif (CRRM) in COVID-19 [6].

FIG. 3 is an alignment of the two COVID CRRM motifs and RNA targets within the 5′untranslated region of the RNA encoding amyloid precursor protein (APP).

FIG. 4 shows the structures of the benzimidazoles JTR-009 (also known as APP Blocker-9) (“9.0”) and BL-1 (prion 5′UTR screened).

FIG. 5 shows the structures of some exemplary analogs of JTR-009.

FIG. 6 shows the structures of the exemplary benzimidazole anthelmintic fenbendazole, oxibendazole, and albendazole.

FIG. 7 shows the structures of exemplary benzimidazole antacids rabeprazole, esomeprazole, and lansoprazole.

FIG. 8 shows structures of paroxetine, Syn-516, A3, B1, vincadifforme, and vinodolinine.

FIGS. 9A-B show that the APP 5′UTR blocker benzimidazole APP blocker-9 (JTR-009) inhibited COVID-19 viral spike protein. 9A: Typical anti-viral efficacy assays conducted for the top COVID-19 inhibitors from a range of APP 5′UTR-directed inhibitors. In this exemplary case, Vero E6 cells were infected with SARS-CoV-2 at a multiplicity of infection of 0.1 and treated either with DMSO (negative control) or compound at the indicated concentrations. At 2 days post infection, cells were fixed and stained with an antibody against SARS-CoV-2 nucleocapsid protein. Cell nuclei were stained with DAPI. Infection rates quantified using Qupath software. 9B: Densitometric quantitation of JTR-009 dependent inhibition of Spike protein in COVID-19 infected Vero E6 cells (based on levels of Spike immunocytochemistry shown).

FIG. 10 is a graph showing the results of a viral plaque assay: Vero-E6 cells were infected with USA-WA1/2020 at a MOI of 0.0001. Drugs added after infection at 1 and 10 uM (Albendazole (Allo), Lansoprazole (Lanso) and JTR-009 (#9)) in plaque assay media. Experimental treatments were in duplicate.

FIGS. 11A-C is a graph showing the results of a viral plaque assay: Vero-E6 cells were infected with USA-WA1/2020 at a MOI of 0.0002. Drugs were added to the overlay media after infection at 0.1, 1, and 10 uM (Lansoprazole (11A), Albendazole (11B),) and JTR-009 (11C)) in plaque assay media. Plaques were counted in duplicate at constant dilutions.

FIG. 12 is a graph showing the results of a viral plaque assay: Vero-E6 cells were infected with USA-WA1/2020 at a MOI of 0.0002. Drugs were added to the overlay media after infection as follows: B1 at 1 uM; Posiphen at 1 uM; Syn-516 at 1 uM; and A3 at 0.5 uM.

### DETAILED DESCRIPTION

As shown herein, a critical RNA motif involved in translational regulation of the amyloid precursor protein (APP) mRNA [1] has strong sequence homology with two sequences in the Covid-19 RNA that are within 5,000 bases on each side of the frame shift sequence critically involved in viral replication [2-5].

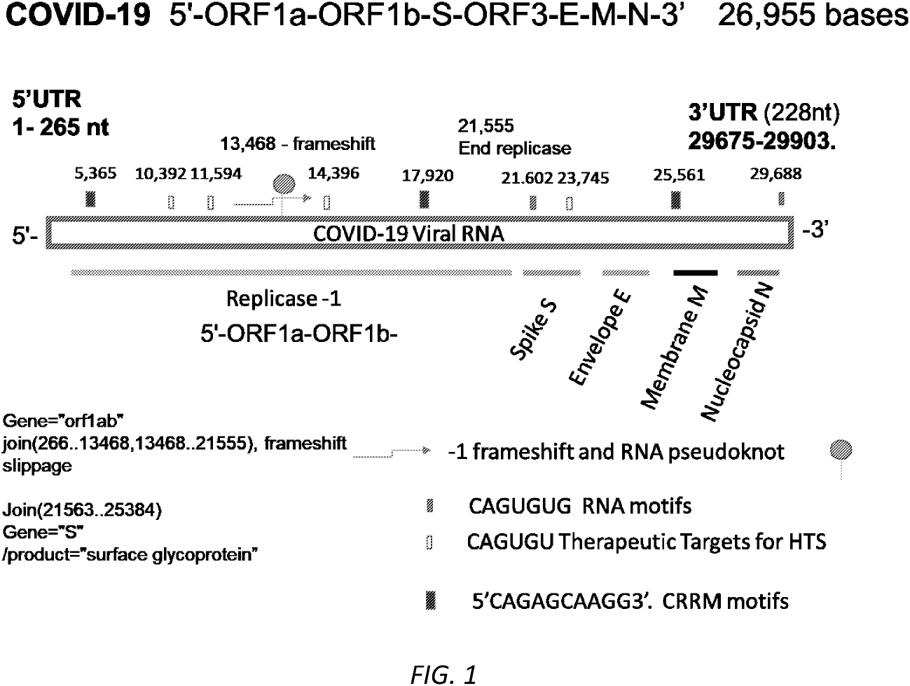

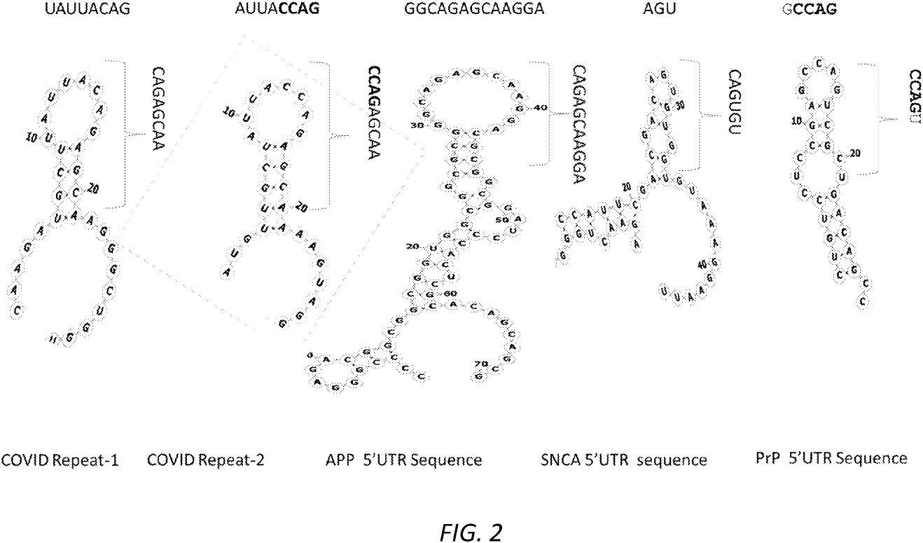

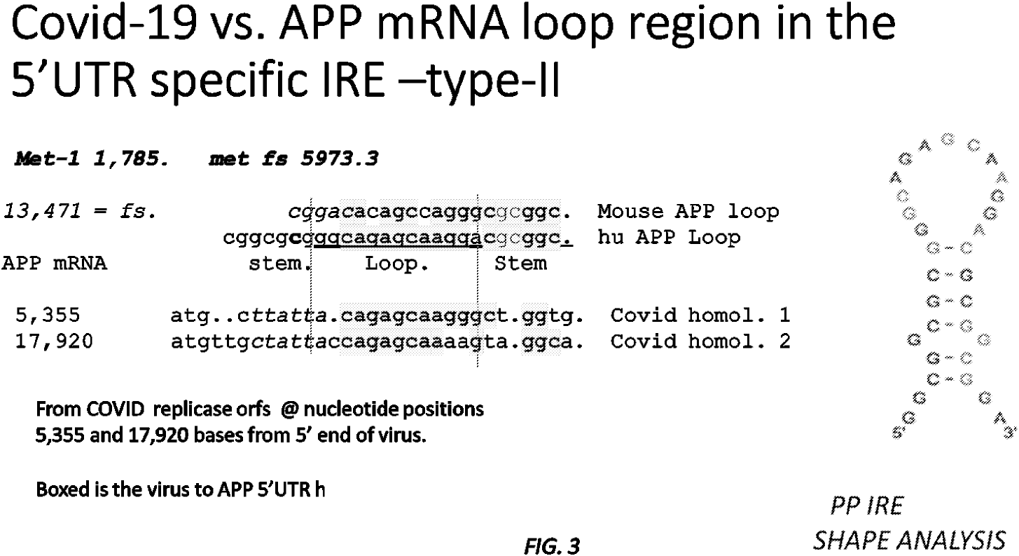

Employing bioinformatics tools, two key related 30-base RNA motifs were found in the replicase transcript on the COVID-19 single-stranded RNA genome (Wuhan sequence of 29,903 bases) (see FIGS. 1-3). These repeats, referred to herein as ‘Covid Replicase Repeat Motifs’ (‘CRRM motifs’), are predicted to form two nearly identical RNA stem loops that are highly related to the CAGUGCAAGG (SEQ ID NO: 1) motif in the loop region of the known Iron-responsive Element-type-II (IRE-type II) in the 5′untranslated region (5′UTR) of APP mRNA [6]. (see FIGS. 1-3).

The first RNA motif CRRM-1 (+5365) was present at a distance 8,103 bases 5′ upstream of the frameshift site (+13,468) in the replicase transcript and the second homologous RNA motif (CRRM-2 @ +17,920) was found 4,452 bases 3′ downstream of this frameshift site (FIGS. 1-3). These motifs were designated as “CRRM-1” and “CRRM-2”, respectively.

In addition to the CRRM motifs, the RNA replicase gene in SARS-CoV-2 contains three CAGUGU (SEQ ID NO:2) motifs that are located in pivotal regulatory regions of its’ RNA genome, on either side of the RNA pseudoknot/frameshift region and in the 3′UTR. These targets were used to identify potent α-Synuclein RNA directed drugs, which are active and are now being validated for use in lowering this pathogenic protein of Parkinson’s disease in dopaminergic neuron cultures as well as for antiviral action towards SARS-CoV-2 [7].

Over the past 10 years, the APP IRE-type-II has been employed as a drug target for characterizing and cataloging both novel and FDA pre-approved drugs that would generate anti-amyloid efficacy for Alzheimer’s disease (AD) and AD-DS (Down’s Syndrome) patients [7].

A panel of previously screened putative RNA-binding small molecule benzimidazoles that were characterized to block APP translation via binding to the CAGAGCAAGG (SEQ ID NO: 1) RNA motif in its’ 5′UTR were screened to identify those that bind to the related repeat CRRM RNA sequences in the viral replicase mRNA of the 30 K base COVID-19 RNA genome [8-10], see, e.g., ref [2] for viral non-structural proteins (NSP) 1 to 16 of COVID-19. In addition, the ability of the compounds to reduce viral activity was assayed in non-human primate-derived VERO cells, which are competent for infection with SARS-CoV2. Agents identified as hits can be used as COVID-19 therapies.

### Methods of Treatment and Prophylaxis

Described herein are methods of treating, or reducing the risk of, infection with SARS-CoV-2 (COVID-19). The methods can be used to treat subjects who have, or have been diagnosed with, infection with SARS-CoV-2 (i.e., COVID-19). The methods can also be used to reduce the risk of infection in a subject who may be, or who may have been, exposed to the SARS-CoV-2 virus. The methods include administering a therapeutically or prophylactically effective amount of an agent described herein.

The present methods include the administration of one or more agents identified herein as blocking translation of the SARS-CoV-2 replicase or as reducing viral infectivity. The agents include benzimidazoles, as well as alkaloids and piperazine compounds.

### Benzimidiazole Agents

A number of benzimidazole agents are described herein that are useful in the present methods, including analogs of JTR-009. In some embodiments, the benzimidazole is an antacid or antihelmintic.

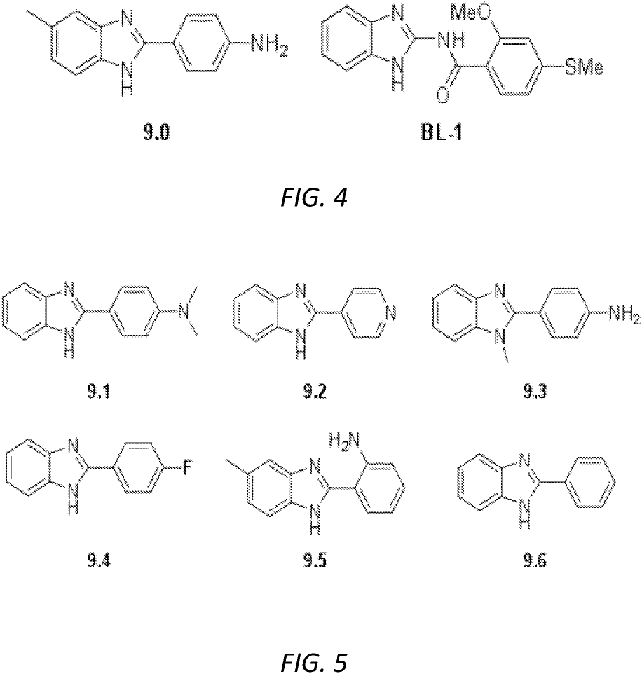

JTR-009 (also known as APP Blocker-9) is a potent APP blocker identified from a screen of APP 5′UTR dependent translation enhancement [25]. As shown herein, JTR-009 showed dose responsive anti-viral activity to prevent CoV2 infection of Vero-C6 cells. Benzimidazole analogs of JTR-009, e.g., as described in WO2014179303, can also be used. See FIGS. 4 and 5.

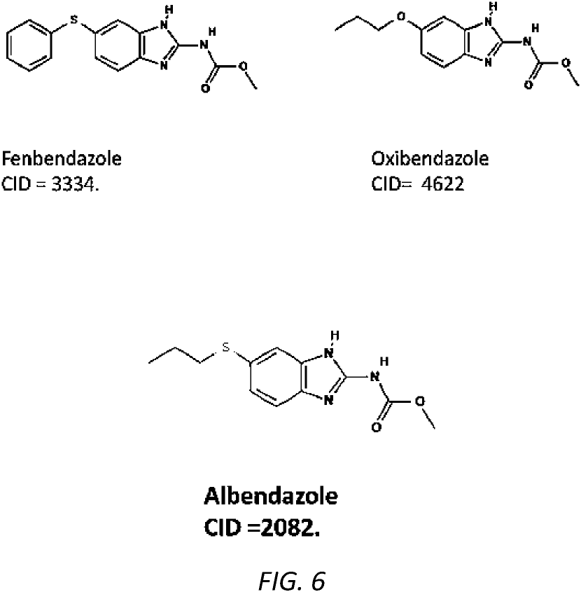

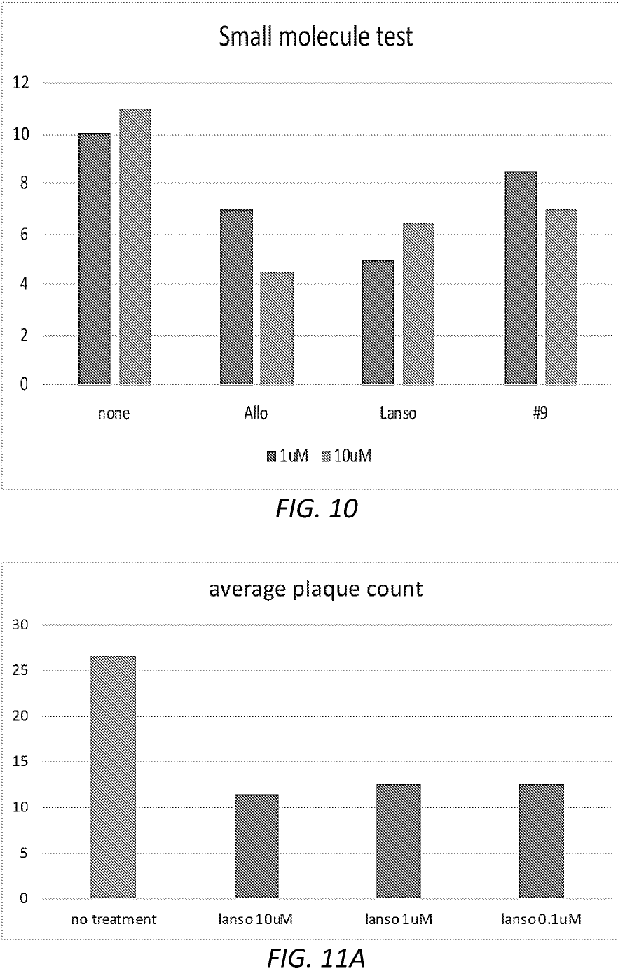

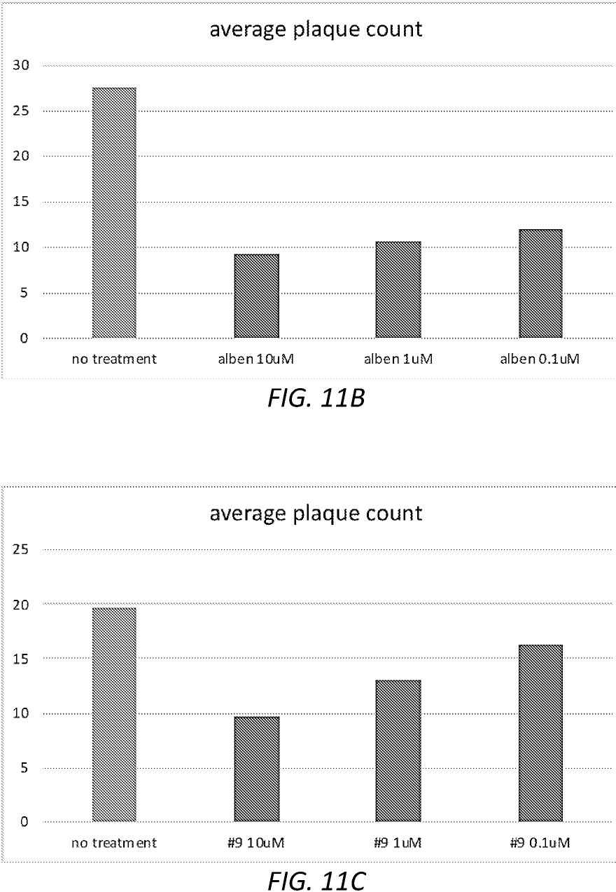

Albendazole, fenbendazole, and oxibendazole are anthelmintic benzimidazole drugs; see FIG. 6. Albendazole exhibited 92.2 % inhibition of APP 5′UTR driven expression of a Luciferase reporter gene in a high-throughput transfection-based assay run in SH-SY5Y cells. Albendazole showed dose responsive anti-viral activity to reduce risk of CoV2 infection of Vero-C6 (African green monkey kidney cells (Vero)) (N=3), FIGS. 10 and 11A-B.

Fenbendazole inhibited APP 5′UTR activity by 91.5% in the above screen, and oxibendazole showed 85.9% inhibition of APP 5′UTR screen activity in the same screen.

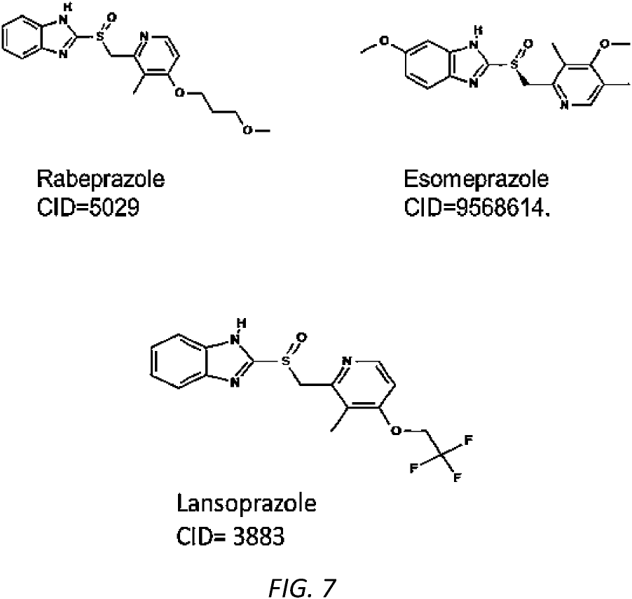

Rabeprazole, omeprazole, esomaprazole, and lansopazole are benzimidazole antacids that are structural analogs of JTR-009; see FIG. 7. Lansopazole, which is clinically used to treat peptic ulcers, showed dose responsive anti-viral activity to prevent CoV2 infection of Vero-C6 (African green monkey kidney cells (Vero)) With Dr. Julie Boucau, Ragon. Inst., MGH), N=3. This agent was predicted to decrease COVID-19 infectivity via the CRRM sequence based on its similarity to JTR-009.

BL-1, a benzimidazole ferritin activator (Rogers et al., Int J Mol Sci. 2019 Feb; 20(4): 994) (see FIG. 4), can also be used.

In some embodiments, a combination of agents is used, e.g., a combination of a benzimidazole agent described herein with desferrioxamine, deferriprone, or a vaccine.

### Additional Agents

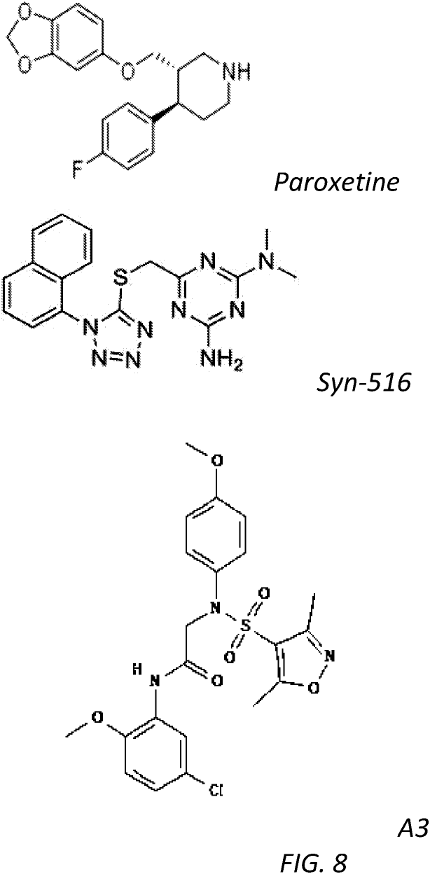

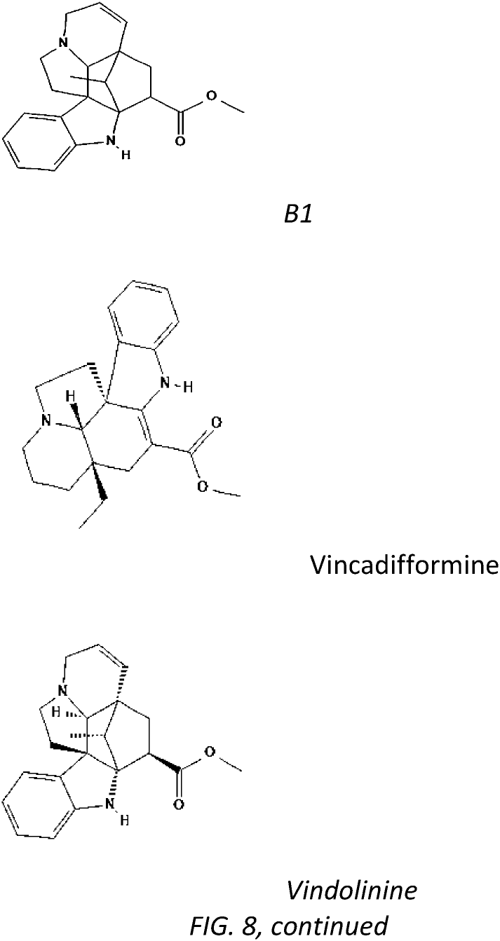

Additional agents that target the alpha-synuclein and /or APP mRNA specific 5′untranslated region and limit asyn and / or translation, and can be used in the methods described herein, include Syn-516 (also known as ML-150, PubChem CID:1517919); A3 (PubChem CID: 3240730, see also US 20090163545); azithromycin; pilocarpine; dimercaptopropanol; tetracycline; paroxetine (the (-)trans isomer of 4-(4 -fluorophenyl)-3-(3′,4 - methylenedioxy-phenoxymethyl)-piperidine, and analogs thereof, including the 3-substituted 1-alkyl-4-phenylpiperidines described in US4007196 and those described in EP0190496A2, EP0223403, EP026657, WO1991009032, and US606392), see FIG. 8; tamsulosin; desferrioxamine (DFO) or deferriprone; phenserine or posiphen (Mikkilineni et al., Parkinsons Dis. 2012;2012:142372); and the αsyn 5′UTR directed vindoline and analogs thereof including a racemic mixture thereof which is referred to herein as B1 (PubChem CID: 120879), vincadifformine, vindolinine (see Ishikawa et al., J. Am. Chem. Soc. 2006, 128, 32, 10596-10612 and FIG. 8), N(1)-methyl-14,15-didehydroaspidofractinine (PubChem CID 372414), and Kopsijasminine (PubChem CID 23626484); and ethyl (1R,9R,16R,17R,18S,21R)-17-hydroxy-2,12-diazahexacyclo[14.2.2.19,12.01,9.03,8.016,21]henicosa-3,5,7,14-tetraene-18-carboxylate (PubChem CID 24770432), and analogs of any of the above. See, e.g., FIG. 8. Other potential compounds include prochlorperazine; diphenylhydramine; netilmycin; mitomycin C; and diptheria toxin (e.g., ONTAK (denileukin diftitox, recombinant diphtheria toxin) and TAGRAXOFUSP (recombinant human interleukin-3 fused to a truncated diphtheria toxin)).

In some embodiments, e.g., where vindoline is used, no other active agent is used. In some embodiments, e.g., where vindoline is used, chloroquine is not used. In some embodiments, a combination of agents is used, e.g., a combination of an agent described herein with desferrioxamine, deferriprone, or a vaccine.

### Pharmaceutical Compositions and Methods of Administration

The methods described herein include the use of pharmaceutical compositions comprising or consisting of an agent described herein as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., desferrioxamine, deferriprone, or a vaccine.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1. APP 5′UTR Blocking Benzimidazoles Inhibit SARS-CoV-2 Infectivity

Addressing our RNA targets in COVID-19, we sought to identify agents among FDA pre-approved benzimidazoles which, like their experimental comparator APP blocker-9 (JTR-009), block the translation of the APP 5′UTR driven expression of a luciferase reporter via its “IRE-type 2” RNA stem loop (AID_1285, Columbia University Genome Center) (see FIGS. 4-8). We characterize each of these agents for their potential to inhibit the replicase complex of ORF1a/b in the COVID-19 RNA genome and to limit viral infectivity. This is predicted to occur by attenuating translation of NSP-3 and NSP14 as the sub-genomic NSPs that encode the two CRRM RNA stem-loops motifs [12].

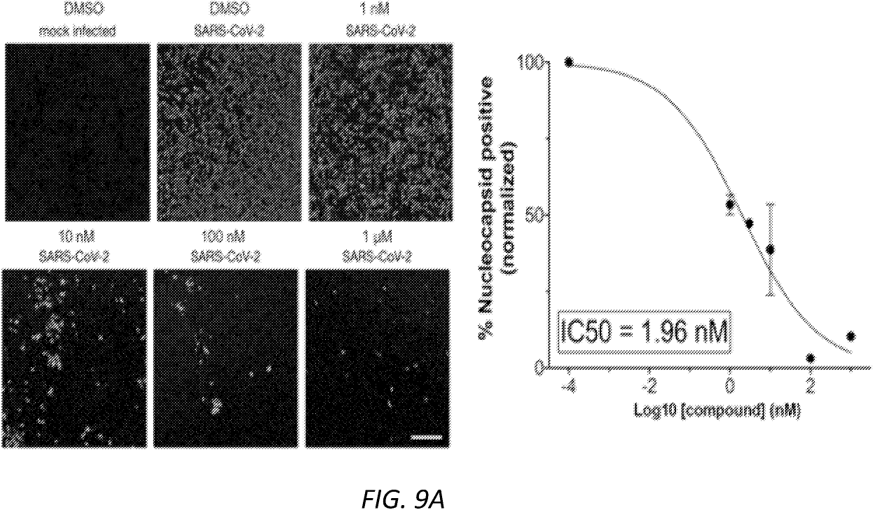

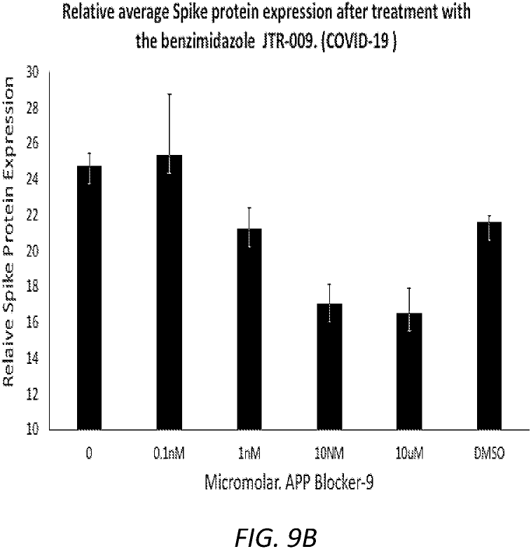

Initial virus assays were performed to experimentally test the benzimidazole APP 5′UTR directed translation blocker-9 (JTR-9) (FIG. 9). JTR-009 reduced APP mRNA translation, prevented binding of the APP 5′UTR to its cognate RNA binding protein, IRP1 to reflect reduced APP expression [25,27]. We tested JTR-009 for possible antiviral efficacy potentially via the related the CRRM motifs in the replicase open reading frame (ORF) related to the APP 5′UTR. We employed immunocytochemical fluorescence of Spike Protein as readout for viral infectivity. This assay indeed demonstrated that JTR-009 dose-dependently inhibited COVID-19 infectivity. Therapeutic activity is being tested via the two CRRM RNA motifs in the virus replicase (FIG. 9).

To characterize the anti-COVID-19 action of the top two benzimidazoles as APP 5′UTR targeting translation blockers, viral plaque assays were performed in which a monolayer of Vero-E6 cells was infected with USA-WA1/2020 at a MOI of 0.0001 or 0.0002. Cell lysis results in colorless contrasting areas (plaques) when the cell monolayer was stained with Crystal violet. To test inhibitors, we set one plate with incrementally increased doses of APP/CRRM directed inhibitors and one without and then compare the number of plaques generated. Plating: regular TC-treated 12-well plates to culture the Vero-E6 cells in DMEM+Pen/ Strep+Glutamax+HEPES+10% FBS. Drugs were added after infection at 1 uM and 10 uM (Albendazole, Lansoprazole and JTR-009) in plaque assay media. Plaques were counted in duplicate at constant dilutions. The results, shown in FIGS. 10 and 11A-C, demonstrate that the small molecules inhibited viral infectivity.

### Example 2. APP 5′UTR Blocking Compounds Inhibit SARS-CoV-2 Infectivity

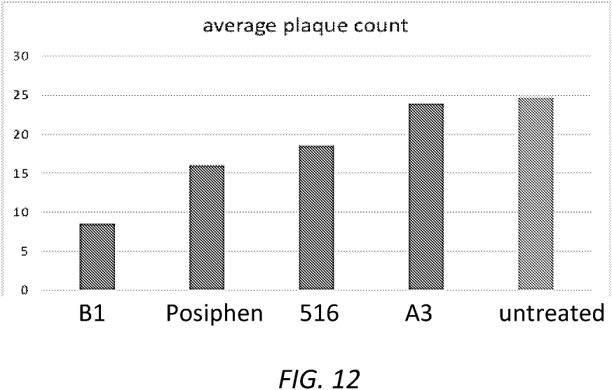

Other small molecules, previously identified as APP 5′UTR targeting translation blockers, were tested in the same viral plaque assays described in Example 1. Vindoline (B1) was tested at 1 uM, posiphen (Pon) was tested at 1 uM, D tested at 1 uM, and A3 was tested at 0.5 uM. The results, presented in FIG. 12, showed that B1; Posiphen; Syn-516; and A3 all reduced viral infectivity.

### Example 3. Benzimidazoles Bind Specifically to SARS-CoV-2 Sequences

The relative binding affinity (Kd) of each of the six APP 5′UTR directed compounds when intercalating into the COVID-19 replicase’s CAGAGCAAGG (SEQ ID NO:1) as encoded in RNA oligos is tested using fluorescence/ thermal assays (Tm calorimetry [21]). Briefly, steady-state fluorescence measurements are performed, e.g., on a Cary Eclipse fluorimeter (Varian) at Rt in 50 mM Tris (pH 7.4)/100 mM NaCl/1 mM MgCl2 on RNA that had been melted and reannealed. The Kd of the inhibitor -CRRM RNA complexes are calculated by curve fitting of the fluorescence intensity as a function of [21] concentration of 10 µM by using the following equation (assuming 1:1 binding): Y= A(1/(([34]/Kd)+ 1)), where A is the difference in fluorescence intensity at 0 µM mRNA and an infinite mRNA concentration. RNA thermal melt of Replicase CAGAGCAAGG RNA is plus/ minus blocker [21]. Binding assays are repeated on ferritin IRE and APP IRE-Type-II RNA-oligonucleotides to ensure specificity.

### References

1 Rogers JT, Cahill CM. 2020. Iron-responsive-like elements and neurodegenerative ferroptosis. Learn Mem 27: 395-413.

2 Yoshimoto FK. 2020. The Proteins of Severe Acute Respiratory Syndrome Coronavirus-2 (SARS CoV-2 or n-COV19), the Cause of COVID-19. Protein J 39: 198-216.

3 Fulzele S, Sahay B, Yusufu I, Lee TJ, et al. 2020. COVID-19 Virulence in Aged Patients Might Be Impacted by the Host Cellular MicroRNAs Abundance/Profile. Aging Dis 11: 509-22.

4 Andrews RJ, Peterson JM, Haniff HS, Chen J, et al. 2020. An in silico map of the SARS-CoV-2 RNA Structurome. bioRxiv.

5 Ursu A, Childs-Disney JL, Andrews RJ, O′Leary CA, et al. 2020. Design of small molecules targeting RNA structure from sequence. Chem Soc Rev 49: 7252-70.

6 Rogers JT, Randall JD, Cahill CM, Eder PS, et al. 2002. An iron-responsive element type II in the 5′-untranslated region of the Alzheimer’s amyloid precursor protein transcript. J Biol Chem 277: 45518-28.

7 Rogers JT, Xia N, Wong A, Bakshi R, et al. 2019. Targeting the Iron-Response Elements of the mRNAs for the Alzheimer’s Amyloid Precursor Protein and Ferritin to Treat Acute Lead and Manganese Neurotoxicity. Int J Mol Sci 20: 994.

8 Hillen HS, Kokic G, Farnung L, Dienemann C, et al. 2020. Structure of replicating SARS-CoV-2 polymerase. Nature 584: 154-6.

9 Freeman MC, Graham RL, Lu X, Peek CT, et al. 2014. Coronavirus replicase-reporter fusions provide quantitative analysis of replication and replication complex formation. J Virol 88: 5319-27.

10 Becares M, Pascual-Iglesias A, Nogales A, Sola I, et al. 2016. Mutagenesis of Coronavirus nsp14 Reveals Its Potential Role in Modulation of the Innate Immune Response. J Virol 90: 5399-414.

11 Khalili JS, Zhu H, Mak NSA, Yan Y, et al. 2020. Novel coronavirus treatment with ribavirin: Groundwork for an evaluation concerning COVID-19. J Med Virol 92: 740-6.

12 Yoshimoto FK. 2021. A Biochemical Perspective of the Nonstructural Proteins (NSPs) and the Spike Protein of SARS CoV-2. Protein J In Press.

13 Jaiswal A, Maurya M, Maurya P, Barthwal MK. 2020. Lin28B Regulates Angiotensin II-Mediated Let-7c/miR-99a MicroRNA Formation Consequently Affecting Macrophage Polarization and Allergic Inflammation. Inflammation In Press

14 Thoms M, Buschauer R, Ameismeier M, Koepke L, et al. 2020. Structural basis for translational shutdown and immune evasion by the Nsp1 protein of SARS-CoV-2. Science 369: 1249-55.

15 Chou WW, Huang CF, Yeh ML, Tsai YS, et al. 2016. MicroRNA let-7g cooperates with interferon/ribavirin to repress hepatitis C virus replication. J Mol Med (Berl) 94: 311-20.

16 Bandyopadhyay S, Huang X, Cho H, Greig NH, et al. 2006. Metal specificity of an iron-responsive element in Alzheimer’s APP mRNA 5′untranslated region, tolerance of SH-SY5Y and H4 neural cells to desferrioxamine, clioquinol, VK-28, and a piperazine chelator. J Neural Transm: 237-47.

17 Rogers JT, Leiter LM, McPhee J, Cahill CM, et al. 1999. Translation of the alzheimer amyloid precursor protein mRNA is up- regulated by interleukin-1 through 5′-untranslated region sequences. J Biol Chem 274: 6421-31.

18 Bandyopadhyay S, Hartley DM, Cahill CM, Lahiri DK, et al. 2006. Interleukin-1alpha stimulates non-amyloidogenic pathway by alpha-secretase (ADAM-10 and ADAM-17) cleavage of APP in human astrocytic cells involving p38 MAP kinase. J Neurosci Res 84: 106-18.

19 Cho HH, Cahill CM, Vanderburg CR, Scherzer CR, et al. 2010. Selective translational control of the Alzheimer amyloid precursor protein transcript by iron regulatory protein-1. J Biol Chem 285: 31217-32.

20 Cahill CM, Rogers JT. 2008. Interleukin-1beta induction of IL-6 is mediated by a novel phosphatidylinositol 3-kinase dependent AKT/Ikappa B kinase alpha pathway targeting activator protein-1. J Biol Chem 283: 25900-12.

21 Reznichenko L, Amit T, Zheng H, Avramovich-Tirosh Y, et al. 2006. Reduction of iron-regulated amyloid precursor protein and beta-amyloid peptide by (-)-epigallocatechin-3-gallate in cell cultures: implications for iron chelation in Alzheimer’s disease. J Neurochem 97: 527-36.

22 Crapper McLachlan DR, Dalton AJ, Kruck TP, Bell MY, et al. 1991. Intramuscular desferrioxamine in patients with Alzheimer’s disease. Lancet 337: 1304-8.

23 Payton S, Cahill CM, Randall JD, Gullans SR, et al. 2003. Drug discovery targeted to the Alzheimer’s APP mRNA 5′-untranslated region: the action of paroxetine and dimercaptopropanol. J Mol Neurosci 20: 267-75.

24 Morse LJ, Payton SM, Cuny GD, Rogers JT. 2004. FDA-Preapproved Drugs Targeted to the Translational Regulation and Processing of the Amyloid Precursor Protein. J Mol Neurosci 24: 129-36.

25 Bandyopadhyay S, Cahill C, Balleidier A, Huang C, et al. 2013. Novel 5′ untranslated region directed blockers of iron-regulatory protein-1 dependent amyloid precursor protein translation: implications for down syndrome and Alzheimer’s disease. PLoS One 8: e65978.

26 Bandyopadhyay S, Huang X, Lahiri DK, Rogers JT. 2010. Novel drug targets based on metallobiology of Alzheimer’s disease. Expert Opin Ther Targets 14: 1177-97.

27 Hamy F, Felder ER, Heizmann G, Lazdins J, et al. 1997. An inhibitor of the Tat/TAR RNA interaction that effectively suppresses HIV-1 replication. Proc Natl Acad Sci U S A 94: 3548-53.

28 Yoshimoto FK. 2021. A Biochemical Perspective of the Nonstructural Proteins (NSPs) and the Spike Protein of SARS CoV-2. Protein J.

29 Rogers JT, Xia N, Wong A, Bakshi R, et al. 2019. Targeting the Iron-Response Elements of the mRNAs for the Alzheimer’s Amyloid Precursor Protein and Ferritin to Treat Acute Lead and Manganese Neurotoxicity. Int J Mol Sci 20:994.

30 Velagapudi SP, Luo Y, Tran T, Haniff HS, et al. 2017. Defining RNA-Small Molecule Affinity Landscapes Enables Design of a Small Molecule Inhibitor of an Oncogenic Noncoding RNA. ACS Cent Sci 3: 205-16.

31 Wu P. 2020. Inhibition of RNA-binding proteins with small molecules. Nat Rev Chem 4: 441-58.

32 Kuo YM, Nwankwo EI, Nussbaum RL, Rogers J, et al. 2019. Translational inhibition of alpha-synuclein by Posiphen normalizes distal colon motility in transgenic Parkinson mice. Am J Neurodegener Dis 8: 1-15.

33 Donahue CP, Ni J, Rozners E, Glicksman MA, et al. 2007. Identification of tau stem loop RNA stabilizers. J Biomol Screen 12: 789-99.

34 Abisambra JF, Fiorelli T, Padmanabhan J, Neame P, et al. 2010. LDLR expression and localization are altered in mouse and human cell culture models of Alzheimer’s disease. PLoS One 5: e8556.

35 Zhang P, Park HJ, Zhang J, Junn E, et al. 2020. Translation of the intrinsically disordered protein alpha-synuclein is inhibited by a small molecule targeting its structured mRNA. Proc Natl Acad Sci U S A 117: 1457-67.

36 Lopez de Silanes I, Galban S, Martindale JL, Yang X, et al. 2005. Identification and functional outcome of mRNAs associated with RNA-binding protein TIA-1. Mol Cell Biol 25: 9520-31.

37 Rogers JT, Venkataramani V, Washburn C, Liu Y, et al. 2016. A role for amyloid precursor protein translation to restore iron homeostasis and ameliorate lead (Pb) neurotoxicity. J Neurochem 138: 479-94.

38 Allerson CR, Cazzola M, Rouault TA. 1999. Clinical severity and thermodynamic effects of iron-responsive element mutations in hereditary hyperferritinemia-cataract syndrome. J Biol Chem 274: 26439-47.

39 Wienken CJ, Baaske P, Duhr S, Braun D. 2011. Thermophoretic melting curves quantify the conformation and stability of RNA and DNA. Nucleic Acids Res 39: e52.

40 Wienken CJ, Baaske P, Rothbauer U, Braun D, et al. 2010. Protein-binding assays in biological liquids using microscale thermophoresis. Nat Commun 1: doi.org/10.1038/ncomms93.

41 Ganesan LP, Mohanty S, Kim J, Clark KR, et al. 2011. Rapid and efficient clearance of blood-borne virus by liver sinusoidal endothelium. PLoS Pathog 7: e1002281.

42 Ganesan LP, Kim J, Wu Y, Mohanty S, et al. 2012. FcgammaRIIb on liver sinusoidal endothelium clears small immune complexes. J Immunol 189: 4981-8.

43 Kim J, Molina RM, Donaghey TC, Buckett PD, et al. 2011. Influence of DMT1 and iron status on inflammatory responses in the lung. Am J Physiol Lung Cell Mol Physiol 300: L659-65.

44 Venkataramani V, Doeppner TR, Willkommen D, Cahill CM, et al. 2018. Manganese causes neurotoxic iron accumulation via translational repression of amyloid precursor protein and H-Ferritin. J Neurochem 147: 831-48.

45 Long JM, Maloney B, Rogers JT, Lahiri DK. 2019. Novel upregulation of amyloid-beta precursor protein (APP) by microRNA-346 via targeting of APP mRNA 5′-untranslated region: Implications in Alzheimer’s disease. Mol Psychiatry 24: 345-63.

46 Bandyopadhyay S, Rogers JT. 2014. Alzheimer’s disease therapeutics targeted to the control of amyloid precursor protein translation: maintenance of brain iron homeostasis. Biochem Pharmacol 88: 486-94.

47 Ross NT, Metkar SR, Le H, Burbank J, Cahill C, Germain A, MacPherson L, Bittker J, Palmer M, Rogers JT, Schreiber SL. ONLINE Probe Report of the Molecular Libraries Network of the NIH, pathway to discovery. Title: Identification of a small molecule that selectively inhibits alpha-synuclein translational expression. 2011. Available at ncbi.nlm.nih.gov/books/NBK55070/.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has an infection with a coronavirus, the method comprising administering an effective amount of a benzimidazole.

2. A method for reducing the risk of infection, severity of infection, with a coronavirus, the method comprising administering an effective amount of a benzimidazole.

3. The method of claim 1, wherein the coronavirus is SARS-CoV-2.

4. The method of claim 1, wherein the coronavirus comprises a sequence comprising CAGUGCAAGG (SEQ ID NO: 3 ) or CAGUGU (SEQ ID NO:2).

5. The method of claim 4, wherein the benzimidazole binds to a sequence comprising CAGUGCAAGG (SEQ ID NO: 3 ) or CAGUGU (SEQ ID NO:2).

6. The method of claim 1, wherein the benzimidazole is an antihelmintic or antacid.

7. The method of claim 6, wherein the benzimidazole antihelmintic is albendazole, fenbendazole, or oxibendazole.

8. The method of claim 6, wherein the benzimidazole antacid is lansoprazole, omeprazole, esomaprazole, or rabeprazole.

9. The method of claim 1, wherein the benzimidazole is JTR-009 or an analog thereof.

10. The method of claim 1, wherein the benzimidazole is BL-1.

11. (canceled)

12. A method for reducing the risk of infection, severity of infection, with a coronavirus, or for treating a subject who has an infection with a coronavirus, the method comprising administering an effective amount of an agent selected from Syn-516; A3; Azithromycin; pilocarpine; dimercaptopropanol; tetracycline; paroxetine; tamsulosin; desferrioxamine (DFO); deferriprone; phenserine or posiphen; vindoline and analogs thereof, optionally B1, vincadifformine, vindolinine, (1)-methyl-14,15-didehydroaspidofractinine, Kopsijasminine, or ethyl (1R,9R,16R,17R,18S,21R)-17-hydroxy-2,12-diazahexacyclo[14.2.2.19,12.01,9.03,8.016,21]henicosa-3,5,7,14-tetraene-18-carboxylate; prochlorperazine; diphenylhydramine; netilmycin; mitomycin C; and diptheria toxin.

13. The method of claim 12, wherein the coronavirus is SARS-CoV-2.

14. The method of claim 12, wherein the coronavirus comprises a sequence comprising CAGUGCAAGG (SEQ ID NO: 3 ) or CAGUGU (SEQ ID NO: 2).

15. The method of claim 14, wherein the benzimidazole binds to a sequence comprising CAGUGCAAGG (SEQ ID NO: 3 ) or CAGUGU (SEQ ID NO:2).

16-30. (canceled)


SEQUENCE LISTING (15 sequences)

SEQ ID NO: 1 - RNA, 10 nt, Severe acute respiratory syndrome-related coronavirus
cagagcaagg

SEQ ID NO: 2 - RNA, 6 nt, Severe acute respiratory syndrome-related coronavirus
cagugu

SEQ ID NO: 3 - RNA, 10 nt, Severe acute respiratory syndrome-related coronavirus
cagugcaagg

SEQ ID NO: 4 - RNA, 30 nt, Severe acute respiratory syndrome-related coronavirus
caagaugcuuauuacagagcaagggcuggu

SEQ ID NO: 5 - RNA, 28 nt, Severe acute respiratory syndrome-related coronavirus
auguugcuauuaccagagcaaaaguagg

SEQ ID NO: 6 - RNA, 70 nt, Homo sapiens
ccccgggagacggcggcgguggcggcgcgggcagagcaaggacgcggcggaucccacucgcacagcagcg

SEQ ID NO: 7 - RNA, 13 nt, Homo sapiens
ggcagugcaagga

SEQ ID NO: 8 - RNA, 11 nt, Homo sapiens
cagugcaagga

SEQ ID NO: 9 - RNA, 31 nt, Homo sapiens
ccauucgacgacagugugguguaaaggaauu

SEQ ID NO: 10 - RNA, 28 nt, Homo sapiens
cuguccuccgagccagucgcugacagcc

SEQ ID NO: 11 - RNA, 21 nt, Mus sp.
cggacacagccagggcgcggc

SEQ ID NO: 12 - RNA, 26 nt, Homo sapiens
cggcgcgggcagagcaaggacgcggc

SEQ ID NO: 13 - DNA, 27 bp, Severe acute respiratory syndrome-related coronavirus
atgcttattacagagcaagggctggtg

SEQ ID NO: 14 - DNA, 30 bp, Severe acute respiratory syndrome-related coronavirus
atgttgctattaccagagcaaaagtaggca

SEQ ID NO: 15 - RNA, 31 nt, Artificial Sequence
  Description of Artificial Sequence: Synthetic oligonucleotide
ggcggcgcgggcagagcaaggacgcggcgga